(19) United States
(12) Patent Application Publication
(10) Pub. No.: US 2020/0375855 A1
(43) Pub. Date: Dec. 3, 2020

(54) ONE-STEP LIQUID LIP COMPOSITION

(71) Applicant: L'ORÉAL, Paris (FR)

(72) Inventors: Tianyi Liu, Springfield, PA (US); Roselin Rosario-Meléndez, New York, NY (US); Trinh Nguyen, Clark, NJ (US)

(73) Assignee: L'ORÉAL, Paris (FR)

(21) Appl. No.: 16/425,099

(22) Filed: May 29, 2019

(51) Int. Cl. A61K 8/06; A61K 8/81; A61K 8/92; A61K 8/25; A61Q 19/00

(52) U.S. Cl. CPC A61K 8/062; A61K 8/81; A61Q 19/001; A61K 8/25; A61K 8/92

(57) ABSTRACT

Disclosed is a one-step lip cosmetic composition and a method for applying the cosmetic composition. The disclosed composition includes an oil phase comprising a silicone resin film former and a silicone-based plasticizer, an aqueous phase comprising an acrylate copolymer resin, a hydrophilic gelling agent and a colorant.

### FIELD OF THE INVENTION

The present invention relates to lip compositions, and specifically to one-step liquid lip products utilizing silicone resin film formers, silicone-based plasticizers, acrylate copolymers, and a hydrophilic gelling agent.

### BACKGROUND

Consumers use lipsticks to cosmetically enhance the appearance of their lips. There is typically a trade-off between durability and aesthetics. Consumers have long desired lipsticks that last for several hours and not transfer to anything they may touch with their lips. These long-lasting lipsticks are typically comprised of a silicone resin, such as MQ resin, and a plasticizing agent. Unfortunately, while durable, these products tend to have tacky feel. As consumer demand increases for higher levels of colorant or metallic finishes, these trade-offs are further exacerbated.

As such, an aesthetic, transfer-resistant composition with high coverage, capable of providing high loading of colorants such as pearlescent or pigments, is therefore highly desirable.

### BRIEF SUMMARY

The present invention is directed to a composition for cosmetic use on lips. The composition includes an oil phase with a silicone resin film former and a silicone-based plasticizer; an aqueous phase with an acrylate copolymer resin; a hydrophilic gelling agent; and a colorant. The silicone resin film former may be a siloxysilicate, such as trimethylsiloxysilicate, and may be present between 2 and 10% w/w. The silicone-based plasticizer may be present at a total amount of between 0.8 and 6% w/w. The hydrophilic gelling agent, which may be a taurate copolymer, may be present at a total amount of between 2 and 7%. The acrylate copolymer resin may be present at a total amount of between 1 and 15%. The colorant, which may be a pigment and/or a pearlescent agent, may be present at a total amount of between 5 and 20%. The emulsion according to claim 1, further comprising at least one volatile alcohol.

The emulsion may also include branched chain hydrocarbons, each having between 10 and 20 carbons, such as isododecane and isohexadecane, at a total amount of between 1 and 15 w/w %. The emulsion may also include a volatile alcohol, which may be present at a total amount of between 1 and 10%.

Also disclosed is a method for making up lips, by applying to the lips a single step lipstick composition comprising from about 2% to about 10% by weight of at least one silicone resin film former in an oil phase, from about 0.8% to about 6% by weight of at least one silicone-based plasticizer in the oil phase, from about 1% to about 15% by weight of an acrylate copolymer resin in an aqueous phase, from about 2% to about 7% by weight of a hydrophilic gelling agent, and from about 5% to about 20% by weight of a colorant.

The applied composition may also include branched chain hydrocarbons, each having between 10 and 20 carbons, such as isododecane and isohexadecane, at a total amount of between 1 and 15 w/w %. The lipstick compositions may also include a volatile alcohol, which may be present at a total amount of between 1 and 10% by weight.

### DETAILED DESCRIPTION

As used herein, articles such as “a” and “an” when used in a claim, are understood to mean one or more of what is claimed or described.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “at least one” means one or more and thus includes individual components as well as mixtures/combinations.

As used herein, the terms “include”, “includes” and “including” are meant to be non-limiting.

The present invention is directed to a one-step liquid lip composition. In preferred embodiments, this is an oil-in-water emulsion. This composition can be applied to lips as a one-step (or single-step) process—that is, the composition can simply be applied to the lips by itself, and no additional composition or work is needed to reduce transferability, or to increase durability.

The composition should comprise five basic components: (1) a silicone resin film former; (2) a silicone-based plasticizer; (3) an acrylate copolymer resin; (4) a hydrophilic gelling agent; and (5) a colorant.

1. Silicone Resin Film Former

The silicone resin film former should be present in an oil phase of the liquid lip composition and should be present in the composition in a total amount of between 2 and 10% by weight, such as between 3 and 7% by weight.

According to preferred embodiments, the long-wear lip compositions of the present invention comprise at least one silicone resin. Examples of suitable silicone resins include those described, for example, in U.S. Pat. Nos. 5,505,937, 5,911,974, 5,965,112, 5,985,298, 6,074,654, 6,780,422, 6,908,621, the disclosures of which are hereby incorporated by reference in their entirety.

According to preferred embodiments, the lip composition contains siloxysilicate resins. One non-limiting example of a siloxysilicate in accordance with the present invention is trimethylsiloxysilicate, which may be represented by the following formula: [(CH₃)₃SiO]ₓ(SiO_{4/2})_{y} wherein x and y may, for example, range from 50 to 80. Such siloxysilicates are commercially available from General Electric, Dow Corning, Wacker, Milliken, Siltech, Grant Industries, Momentive and Shin-Etsu Silicones under the tradename Resin MQ®.

2. Silicone-Based Plasticizer

The silicone-based plasticizer should be present in an oil phase of the liquid lip composition and should be present in the composition in a total amount of between 0.8 and 6% by weight, such as between 2 and 4% by weight.

According to preferred embodiments, the lip composition contains silsesquioxane resins such as, for example, polypropyl silsesquioxane resin.

Silsesquioxane resins are a specific form of silicone resin. Silicone resin nomenclature is known in the art as “MDTQ” nomenclature, whereby a silicone resin is described according to the various monomeric siloxane units which make up the polymer. Each letter of “MDTQ” denotes a different type of unit. When the film-forming resin is made up predominantly of tri-functional units (or T units), it is generally called a silsesquioxane resin, which is described, for example in US 2006/0292096, herein incorporated by reference in its entirety.

Examples of silsesquioxane resins that may be used in the present invention are alkyl silsesquioxane resins that are silsesquioxane homopolymers and/or copolymers having an average siloxane unit of the general formula R¹ₙSiO_{(4-n)/2}, wherein each R¹ is a propyl group, wherein more than 80 mole % of R¹ represent a C₃-C₁₀ alkyl group, n is a value of from 1.0 to 1.4, and more than 60 mole % of the copolymer comprises R¹ SiO_{3/2} units. As each R¹ is a propyl group these polymers are called polypropylsilsesquioxane resins or “t-propyl” silsesquioxane resins. These resins and methods of making them are described, for example in U.S. Pat. Nos. 8,586,013, 8,025,869, 2012/0301415, and 2007/0093619, all of which are herein incorporated by reference in their entirety.

A non-limiting example of a polypropylsilsesquioxane resin suitable for use in the present invention is commercially available from Dow Corning as DOWSIL™ 670 Fluid or DOWSIL™ 680 ID Fluid. These Dow Corning resins have a general formula of RₙSiO_{(4-n)/2} wherein R is independently chosen from a hydrogen atom and a monovalent hydrocarbon group comprising 3 carbon atoms, wherein more than 80 mole % of R are propyl groups, n is a value from 1.0 to 1.4, more than 60 mole % of the copolymer comprises R SiO_{3/2} units, and having a hydroxyl or alkoxy content from 0.2 to 10% by weight, for example between 1 and 4% by weight, preferably between 5 and 10% by weight, and more preferably between 6 and 8% by weight. In certain embodiments, the polypropylsilsesquioxane resin has a molecular weight from about 5,000 to about 30,000 and a T_{g} from about −5° C. to about 5° C.

Other, suitable silicone-based plasticizers include those usually used in the field of application and those which can be a solvent for the copolymer. Examples of silicone plasticizers for purposes of the present invention include phenylsilicones, for instance phenyl trimethicones, phenyl dimethicones, phenyl trimethylsiloxydiphenylsiloxanes, diphenyl dimethicones, diphenyl methyldiphenyl trisiloxanes, 2-phenylethyl trimethylsiloxysilicates, trimethyl pentaphenyl trisiloxane, tetramethyl hexaphenyl trisiloxane and trimethyl pentaphenyl trisiloxane.

3. Acrylate Copolymer Resin

The acrylate copolymer resin should be present in an aqueous phase of the liquid lip composition and should be present in the composition in a total amount of between 2 and 7%.

Suitable acrylates copolymers may be dispersed in water, and may include acrylic copolymer dispersions sold under the names Neocryl® XK-90 (acrylic/styrene copolymer), Neocryl A-1070® (acrylic/styrene copolymer), Neocryl A-1090® (acrylic/styrene copolymer), Neocryl BT-62® (acrylic/styrene copolymer), Neocryl A-1079® (acrylic/styrene copolymer) and Neocryl A-523® (acrylic/styrene copolymer) by the company Avecia-Neoresins; NeoCryl® XK-320 (acrylic styrene copolymer emulsion), and NeoCryl® A-1120 (modified acrylic/styrene copolymer dispersion) by the company DSM; Dow Latex 432® (styrene/acrylates copolymer) by the company Dow Chemical; Syntran® PC 5620 (styrene/acrylates/ammonium/methacrylate copolymer (and) sodium lauryl sulfate (and) sodium laureth sulfate) by the company Interpolymer; Rheoplex P376 (acrylic copolymer emulsion) by the company Dow Chemical; Daitosol 5000 AD® (acrylates copolymer) by the company Daito Kasey Kogyo; Allianz™ OPT (acrylates/C12-C22 alkylmethacrylate copolymer) by ISP; and Epitex 66 (acrylates copolymer) by the company Dow Chemical.

In some embodiments, the acrylates copolymer has a weight average molecular weight ranging from about 75,000 to 140,000 g/mol, preferably ranging from about 84,000 to 125,000 g/mol, and most preferably ranging from about 88,000 to 120,000 g/mol, and a T_{g} ranging from about −20 to 50° C., preferably from about −10 to 40° C., and most preferably from about 0 to 20° C. A particularly preferred acrylates copolymer for use in the present invention is one having a weight average molecular weight of from about 93,000 to 114,000 g/mol, and a T_{g} of about 13.6° C., sold under the tradename EPITEX™ 66 Polymer by Dow Chemical in the form of an aqueous polyacrylate emulsion.

Preferred embodiments of the acrylate copolymer resin are not silicone acrylate copolymers.

4. Hydrophilic Gelling Agent

The hydrophilic gelling agent may be present in either an oil phase or aqueous phase (or both) and should be present in the composition in a total amount of between 1 and 15% by weight. In certain embodiments, the hydrophilic gelling agent is soluble in water and/or in the aqueous phase. In preferred embodiments, the hydrophilic gelling agent comprises a taurate copolymer.

The gelling agent may more particularly be chosen from acrylic polymers described as follows:

4.A. Hydrophilic Acrylic Polymers

According to the invention, the term “hydrophilic acrylic polymers” especially means non-hydrophobic and non-amphiphilic acrylic polymers. Hydrophilic acrylic polymers may include either polyacrylamidomethylpropanesulfonic acid (AMPS®) acrylic polymers or acrylic acid polymers.

Among the hydrophilic acrylic polymers that may be mentioned are (1) Acrylic Polymers Comprising at Least One Monomer Bearing a Sulfonic Group, (2) Acrylamide/AMPS® Copolymers, and (3) Other Hydrophilic Acrylic Polymers.

4.A.1. Acrylic Polymers Comprising at Least One Monomer Bearing a Sulfonic Group

According to a first embodiment, the hydrophilic acrylic polymer may comprise at least one monomer bearing a sulfonic group.

The polymers used in accordance with the invention are homopolymers that may be obtained from at least one ethylenically unsaturated monomer bearing a sulfonic group, which may be in free form or partially or totally neutralized form.

Preferentially, the polymers in accordance with the invention are partially or totally neutralized with a mineral base (sodium hydroxide, potassium hydroxide or aqueous ammonia) or an organic base such as monoethanolamine, diethanolamine, triethanolamine, an aminomethylpropanediol, N-methylglucamine, basic amino acids, for instance arginine and lysine, and mixtures of these compounds. They are generally neutralized.

In the present invention, the term “neutralized” means polymers that are totally or virtually totally neutralized, i.e. at least 90% neutralized.

The polymers used in the composition of the invention generally have a number-average molecular weight ranging from 1,000 to 20,000,000 g/mol, preferably ranging from 20,000 to 5,000,000 g/mol and even more preferentially from 100,000 to 1,500,000 g/mol.

These polymers according to the invention may be crosslinked or noncrosslinked.

The monomers bearing a sulfonic group of the polymer used in the composition of the invention are especially chosen from vinylsulfonic acid, styrenesulfonic acid, (meth)acrylamido(C₁-C₂₂)alkylsulfonic acids, N—(C₁-C₂₂)alkyl(meth)acrylamido(C₁-C₂₂)alkylsulfonic acids such as undecylacrylamidomethanesulfonic acid, and also partially or totally neutralized forms thereof, and mixtures thereof.

According to one preferred embodiment of the invention, the monomers bearing a sulfonic group are chosen from (meth)acrylamido(C₁-C₂₂)alkylsulfonic acids, for instance acrylamidomethanesulfonic acid, acrylamidoethanesulfonic acid, acrylamidopropanesulfonic acid, 2-acrylamido-2-methylpropanesulfonic acid, 2-methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid and 2-acrylamido-2,6-dimethyl-3-heptanesulfonic acid, and also partially or totally neutralized forms thereof, and mixtures thereof.

More particularly, 2-acrylamido-2-methylpropanesulfonic acid (AMPS®), and also partially or totally neutralized forms thereof, is used.

When the polymers are crosslinked, the crosslinking agents may be chosen from the polyolefinically unsaturated compounds commonly used for crosslinking polymers obtained by free-radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl(meth)acrylate, allylic ethers of alcohols of the sugar series, or other allylic or vinyl ethers of polyfunctional alcohols, and also the allylic esters of phosphoric and/or vinylphosphonic acid derivatives, or mixtures of these compounds.

According to one preferred embodiment of the invention, the crosslinking agent is chosen from methylenebisacrylamide, allyl methacrylate and trimethylolpropane triacrylate (TMPTA). The degree of crosslinking generally ranges from 0.01 mol % to 10 mol % and more particularly from 0.2 mol % to 2 mol % relative to the polymer.

The homopolymer of monomers bearing a sulfonic group may be crosslinked with one or more crosslinking agents.

These homopolymers are generally crosslinked and neutralized, and they may be obtained according to the preparation process comprising the following steps:

(a) the monomer such as 2-acrylamido-2-methylpropanesulfonic acid in free form is dispersed or dissolved in a solution of tert-butanol or of water and tert-butanol;

(b) the monomer solution or dispersion obtained in (a) is neutralized with one or more mineral or organic bases, preferably aqueous ammonia NH₃, in the amount making it possible to obtain a degree of neutralization of the sulfonic acid functions of the polymer ranging from 90% to 100%;

(c) the crosslinking monomer(s) are added to the solution or dispersion obtained in (b);

(d) a standard free-radical polymerization is performed in the presence of free-radical initiators at a temperature ranging from 10 to 150° C.; the polymer precipitates in the tert-butanol-based solution or dispersion.

The preferred AMPS® homopolymers are generally characterized in that they comprise, randomly distributed:

(a) from 90% to 99.9% by weight of units of general formula below:

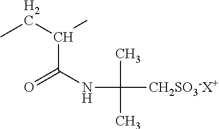

in which X⁺ denotes a proton, an alkali metal cation, an alkaline-earth metal cation or the ammonium ion, not more than 10 mol % of the cations X⁺ possibly being protons H⁺;

(b) from 0.01% to 10% by weight of crosslinking units derived from at least one monomer containing at least two olefinic double bonds; the weight proportions being defined relative to the total weight of the polymer.

The homopolymers according to the invention that are more particularly preferred comprise from 98% to 99.5% by weight of units of formula (II) and from 0.2% to 2% by weight of crosslinking units.

A polymer of this type that may especially be mentioned is the crosslinked and neutralized 2-acrylamido-2-methylpropanesulfonic acid homopolymer sold by the company Clariant under the trade name Hostacerin® AMPS (CTFA name: ammonium polyacryldimethyltauramide).

4.A.2. Acrylamide/AMPS® Copolymers

According to another embodiment, the hydrophilic acrylic polymer is a crosslinked anionic copolymer formed from units derived from the reaction between (i) acrylamide (monomer 1), (ii) 2-acrylamido-2-methylpropanesulfonic acid (monomer 2, referred to hereinbelow for convenience as AMPS®) and (iii) at least one polyolefinically unsaturated compound (monomer 3), constituting here the crosslinking agent.

The above copolymers may thus be obtained conventionally according to the emulsion polymerization technique from three different comonomers included in their constitution.

The polyolefinically unsaturated monomers used as crosslinking agents for the preparation of the copolymers in accordance with the invention are preferably chosen from the group formed by methylenebisacrylamide, allyl sucrose and pentaerythritol. Even more preferentially, use is made of methylenebisacrylamide.

Preferably, said polyolefinically unsaturated compound is present in the copolymer in a concentration of between 0.06 and 1 mmol per mole of the monomer units as a whole.

The ratio, expressed in mol %, between acrylamide and AMPS® is preferentially between 85/15 and 15/85, advantageously between 70/30 and 30/70, even more preferentially between 65/35 and 35/65 and even more particularly between 60/40 and 40/60. In addition, AMPS is generally at least partially neutralized in the form of a salt, for example with sodium hydroxide, with potassium hydroxide or with a low molecular weight amine such as triethanolamine, or mixtures thereof.

Preferred crosslinked copolymers include the products sold under the names Sepigel 305 (CTFA name: polyacrylamide/C13-14 isoparaffin/Laureth 7) or Simulgel 600 (CTFA name: acrylamide/sodium acryloyldimethyltauratecopolymer/isohexadecane/polysorbate 80) sold by the company SEPPIC, or Simulgel EG (CTFA name: sodium acrylate/sodium acryloyldimethyltauratecopolymer/isohexadecane/polysorbate 80).

4.A.3. Other Hydrophilic Acrylic Polymers

As other hydrophilic acrylic polymers that may be used according to the invention, mention may also be made of:

(a) homopolymers or copolymers of acrylic or methacrylic acids or salts thereof and esters thereof, such as the products sold under the names Carbopol 934, 940, 954, 981 and 980 by the company Noveon, Synthalen L® from the company 3V, sodium polymethacrylate sold under the name Darvan No. 7® by the company Vanderbilt, the products sold under the names Versicol F or Versicol K by the company Allied Colloid, Ultrahold 8 by the company Ciba Geigy and polyacrylic acids of Synthalen K type,

(b) polyacrylates and polymethacrylates such as glyceryl acrylate polymers, and in particular copolymers of glyceryl acrylate and of acrylic acid, such as the products sold under the names Lubrajel® MS, Lubrajel® CG, Lubrajel® DV, Lubrajel® NP, Lubrajel® Oil, Lubrajel® Oil BG, Lubrajel® PF, Lubrajel® TW and Lubrajel® WA by the company Guardian Laboratories. Use is preferably made of Lubrajel® MS,

(c) polyacrylic acid/alkyl acrylate copolymers of Pemulen type,

(d) copolymers of acrylic acid salt/vinyl alcohol, such as the product sold under the name Hydragen FN® from Cognis,

(e) and mixtures thereof.

Preferred hydrophilic gelling agents include:

(1) AMPS® and acrylamide copolymers of the Sepigel® or Simulgel® type sold by the supplier Seppic; and

(2) copolymers of AMPS® and polyoxyethylene alkyl methacrylates (optionally cross-linked), and mixtures thereof such as ammonium acryloyldimethyltaurate/steareth-25 methacrylate crosspolymer, available under the tradenames Aristoflex HMS; ammonium acryloyldimethyltaurate/steareth-8 methacrylate crosspolymer, available under the tradenames Aristoflex SNC; and ammonium acryloyldimethyltaurate/VP copolymer, available under the tradenames Aristoflex AVC, Aristoflex JQD, Hostacerin SAF, all commercially available from the supplier Clariant.

Colorants

According to embodiments of the present application, compositions comprising at least one colorant are provided. In certain embodiments, the colorant is a pigment, a pearlescent agent, or a combination thereof. The combined colorants should be present in a total amount of between 5 and 20% by weight.

Suitable colorants include, but are not limited to, lipophilic dyes, pigments and pearlescent agents, and their mixtures. Any colorant typically found in lipstick compositions can be used.

Suitable examples of fat-soluble dyes are, for example, Sudan red, DC Red 17, DC Green 6, β-carotene, soybean oil, Sudan brown, DC Yellow 11, DC Violet 2, DC Orange 5 and quinoline yellow.

Suitable pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

Suitable pearlescents may also be included, and may be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

Color additives, such as natural extracts, may also be appropriate in various embodiments. One such example is spirulina paltensis extract, although other extracts may also be appropriate.

Other Materials

In certain embodiments, the composition's oil phase also includes branched chain hydrocarbons. In some embodiments, each branched chain hydrocarbon has between 10 and 20 carbons. In certain embodiments, the oil phase comprises at least two branched hydrocarbons, while in other embodiments, the oil phase has only two branched hydrocarbons. In some embodiments, the branched chain hydrocarbons include isododecane and isohexadecane. When present, the branched chain hydrocarbons should be present in the composition in a total amount of between 1 and 15% by weight.

In certain embodiments, the composition may also include at least one volatile alcohol, including but not limited to linear or branched lower monoalcohols having 2 to 5 carbon atoms, such as methanol, ethanol, isopropanol or n-propanol, and may be present in a total amount of between 1 and 10% by weight.

This invention is directed to an emulsion cosmetic composition which can be applied with a cooling sensation and may offer a transfer-resistant metallic finish with high coverage. The combination of materials provides a transparent and flexible thin film, which also allows loading of high pearls and/or high pigment.

Other cosmetically acceptable ingredients, such as dimethicone, glycerin, etc., may also be incorporated.

Example Formulations

Referring to the compositions listed in Tables 1 and 2, the pigment was first pre-dispersed in the silicone resin film former and a portion of the branched chain hydrocarbons using a homogenizer. The rest of the ingredients were then combined into the pigment dispersion to form an emulsion by mixing at room temperature. The pearls were added slowly while mixing at room temperature to form the final composition. The composition was then transferred into a desired container.

TABLE 1
Disclosed Formulations

| | Formula 1 | Formula 2 | Formula 3 |
|---|---|---|---|
| Material | % w/w | % w/w | % w/w |
| Silicone Resin Film Former | 2-10% | 2-10% | 2-10% |
| Branched Chain Hydrocarbons | 2-7% | 2-7% | 2-7% |
| Hydrophilic Gelling Agent | 1-5% | 1-5% | 1-5% |
| Silicone-Based Plasticizer | 0.8-6% | 0.8-6% | 0.8-6% |
| Water | 40-60% | 40-60% | 40-60% |
| Volatile Alcohol | 2-7% | 2-7% | 2-7% |
| Acrylate Copolymer Resin | 1-15% | 1-15% | 1-15% |
| Pigment | 0.1-2% | 0.1-2% | 0.1-2% |
| Pearls | 0.1-15% | 0.1-10% | 0.1-5% |

TABLE 2
Comparative Formulations

| | Formula 4 | Formula 5 |
|---|---|---|
| Material | % w/w | % w/w |
| Silicone Resin Film Former | 2-10% | 2-10% |
| Branched Chain Hydrocarbons | 2-7% | 2-7% |
| Hydrophilic Gelling Agent | 1-5% | 1-5% |
| Silicone-Based Plasticizer | 0.8-6% | 0% |
| Water | 40-60% | 40-60% |
| Volatile Alcohol | 2-7% | 2-7% |
| Acrylate Copolymer Resin | 0% | 1-15% |
| Pigment | 0.1-2% | 0.1-2% |
| Pearls | 0.1-10% | 0.1-5% |

Evaluations

The formulations were evaluated based on contact angle, transferring multiple layers, transferring single layers, brittleness, zero shear viscosity, and emulsion droplet size of the formulations without the pearls. The results are summarized in Table 3.

TABLE 3
Summary of Evaluations

| Evaluation | Formula 1 | Formula 2 | Formula 3 | Formula 4 | Formula 3 |
|---|---|---|---|---|---|
| Contact Angle (°) | 110.60 ± | 117.7 ± | 111.7 ± | 98.78 ± | 98.78 ± |
| | 3.76 | 1.31 | 0.85 | 5.62 | 5.62 |
| Transfer-multiple layers | 3% | 3% | 1.52% | 4.15% | 1.53% |
| Transfer-single layer | <1% | <1% | 1.04% | 0.93% | <1% |
| Brittleness | 1 | 0 | 0 | 3 | 2 |
| Zero Shear | 9.80 × 10³ | 9.49 × 10³ | 7.38 × 10³ | 7.42 × 10³ | 1.14 × 10⁴ |
| Viscosity (Pa · s) | | | | | |
| Emulsion droplet | 5-50 μm | 5-50 μm | 5-50 μm | 5-40 μm | 5-100 μm |
| size (no Pearl) | | | | | |

Contact Angle

To characterize the hydrophobicity of each formula when casted into films, contact angle measurement was performed using an Attension® tensiometer and analyzing software. Each formula was first casted onto a drawdown paper (e.g., Black Scrub Panel P121-10N) using a 3 mL drawdown bar, and air dried under room temperature overnight. The coated drawdown paper was then placed onto a moving stage 1 cm below a water dispenser. A 3 μL water droplet was dispensed onto the film, and contact angle was measured as the angle between the surface tension vector of film-water and the surface tension vector of water-air after 10 seconds of equilibration. 3 repeating trails were done on each formula and the average angle and standard deviation were recorded.

Transfers

To quantify multiple layer transfer, approximately 20 mg of each composition were first dispensed onto a 2 cm×2 cm bio-skin square, recording the actual mass. Depending on the product viscosity, the application process requires either 2 or 3 layers. The bio-skin piece was then dried at room temperature for 10 minutes. A separate bio-skin was pressed onto the coated one, while applying a 1 kg weight. The mass transferred was then measured, and a percentage transferred was calculated by dividing the mass transferred by the original mass.

Similarly, for quantifying single layer transfer, approximately 10 mg of each composition was deposited onto a 2 cm×2 cm bio-skin square in a single layer, recording the actual mass. The bio-skin piece was dried at room temperature for 10 minutes. A separate bio-skin was pressed onto the coated one, while applying a 1 kg weight. The mass transferred was then measured, and a percentage transferred was calculated by dividing the mass transferred by the original mass.

Brittleness

To measure brittleness, each formula was deposited onto a drawdown paper (e.g., Black Scrub Panel P121-10N) using a 3 mL drawdown bar. The film was dried at room temperature for 12 hrs. The film-covered drawdown paper was then folded 180° to assess degree of cracking and peel off around the folded line. Scoring of brittleness is described in Table 4 below.

TABLE 4
Brittleness Scoring Standards

| Score | Description |
|---|---|
| 0 | No crack at all, folded |
| | line fully covered |
| 1 | Minor crack along folded |
| | line, no flaking off |
| 2 | Obvious crack along folded |
| | line, some flaking off |
| 3 | Major crack along and around |
| | folded line, major flaking off |

As can be seen, all of the test formulas were highly flexible, scoring either 0 or 1 in brittleness, while the comparative formulas were significantly more brittle. This was surprising, especially the brittleness seen in comparative formula 4.

Zero Shear Viscosity

To measure zero shear viscosity, approximately 1 gram of each composition was first deposited onto the bottom plate of a rheometer. A 40mm flat plate was used as a rheology probe with a gap of 1000 μm between the bottom plate and the probe. Each sample was first equilibrated at 25° C. for 20 seconds, and then a shear rate flow experiment was performed. The duration of experiment is 10 minutes, shear rate changes from 0.001-1000/s, with 5 data points recorded within each decade. After the experiment, a zero-shear viscosity was determined from a log(viscosity) vs. log(shear rate) plot, by linear fitting the initial plateau region to intersect with y-axis. This value represents the viscosity of each formula under unperturbed situation.

Emulsion Droplet Size (No Pearl)

The change of formula's influence on emulsion formation is assessed by taking a microscope image of each formula without pearl (existence of pearl reduces the visibility of droplets) to analyze and measure the oil droplet size range within the continuous water phase. Samples were prepared by combining oil phase and pigment into a high-speed mixer cup and mixing for 5 minutes at 2350 rpm, then adding water phase to the high-speed mixer cup, and mixing for 5 minutes at 2350 rpm. Samples were then taken and placed under a microscope, the image was captured, and average droplet size was then estimated.

Those skilled in the art will recognize or be able to ascertain using no more than routine experimentation, many equivalents to the specific embodiments of the invention described herein. Such equivalents are intended to be encompassed by the following claims.

1. An emulsion for use on lips, comprising:
an oil phase including at least one silicone resin film former and at least one silicone-based plasticizer;
an aqueous phase including an acrylate copolymer resin;
a hydrophilic gelling agent; and
a colorant.

2. The emulsion according to claim 1, wherein the oil phase further comprises a plurality of branched chain hydrocarbons, each having between 10 and 20 carbons.

3. The emulsion according to claim 2, wherein the plurality of branched hydrocarbons consists of isododecane and isohexadecane.

4. The emulsion according to claim 3, wherein the isohexadecane is present in the emulsion in a total amount of between 1 and 15 w/w %.

5. The emulsion according to claim 1, wherein the silicone resin film former is present in the emulsion in a total amount of between 2 and 10%.

6. The emulsion according to claim 1, wherein the silicone resin film former is a siloxysilicate.

7. The emulsion according to claim 6, wherein the siloxysilicate is trim ethylsiloxysilicate.

8. The emulsion according to claim 1, wherein the silicone-based plasticizer is present in the emulsion in a total amount of between 0.8 and 6%.

9. The emulsion according to claim 1, wherein the hydrophilic gelling agent is present in the emulsion in a total amount of between 2 and 7%.

10. The emulsion according to claim 1, wherein the hydrophilic gelling agent comprises a taurate copolymer.

11. The emulsion according to claim 1, wherein the acrylate copolymer resin is present in the emulsion in a total amount of between 1 and 15%.

12. The emulsion according to claim 1, wherein the colorant is a pigment, a pearlescent agent, or a combination thereof.

13. The emulsion according to claim 12, wherein the colorant is present in a total amount of between 5 and 20%.

14. The emulsion according to claim 1, further comprising at least one volatile alcohol.

15. The emulsion according to claim 14, wherein the at least one volatile alcohol is present in the emulsion in a total amount of between 1 and 10%.

16. A method of making up lips comprising applying to the lips a single step lipstick composition comprising:
from about 2% to about 10% by weight of at least one silicone resin film former in an oil phase;
from about 0.8% to about 6% by weight of at least one silicone-based plasticizer in the oil phase;
from about 1% to about 15% by weight of an acrylate copolymer resin in an aqueous phase;
from about 2% to about 7% by weight of a hydrophilic gelling agent; and
from about 5% to about 20% by weight of a colorant.

17. The method according to claim 16, wherein the oil phase further comprises a plurality of branched chain hydrocarbons, each having between 10 and 20 carbons.

18. The method according to claim 17, wherein the plurality of branched hydrocarbons consists of isododecane and isohexadecane.

19. The method according to claim 16, wherein the lipstick composition further comprises from about 1% to about 10% by weight of at least one volatile alcohol.

20. The emulsion according to claim 1, wherein the emulsion comprises:
from about 2% to about 10% by weight of the at least one silicone resin film former;
from about 0.8% to about 6% by weight of at least one silicone-based plasticizer in the oil phase;
from about 1% to about 15% by weight of the acrylate copolymer resin in an aqueous phase;
from about 2% to about 7% by weight of the hydrophilic gelling agent; and
from about 5% to about 20% by weight of the colorant.
